(19) United States
(12) Patent Application Publication
(10) Pub. No.: US 2026/0014084 A1
(43) Pub. Date: Jan. 15, 2026

(54) STABLE FORMULATIONS WITH REDUCED SOLID STATE REACTIONS

(71) Applicant: Applied Materials, Inc., Santa Clara, CA (US)

(72) Inventors: Neha GUPTA, Mumbai (IN); Balaji GANAPATHY, Maharashtra (IN); Sujit DEBNATH, West Bengal (IN); Ankur KADAM, Maharashtra (IN)

(21) Appl. No.: 19/266,994

(22) Filed: Jul. 11, 2025

Related U.S. Application Data
(60) Provisional application No. 63/670,060, filed on Jul. 11, 2024.

(51) Int. Cl. A61K 9/50

(52) U.S. Cl. CPC A61K 9/501; A61K 9/5089

(57) ABSTRACT

The disclosure is directed at a stable pharmaceutical composition comprising a coated ingredient particle, and the use thereof for reducing solid state reactions. Methods for manufacturing such an stable pharmaceutical composition are also provided.

### CROSS-REFERENCE TO RELATED APPLICATIONS

This application claims priority to U.S. Application Ser. No. 63/670,060, filed on Jul. 11, 2024. The disclosure of the prior application is considered part of the disclosure of this application, and is incorporated in its entirety into this application.

### TECHNICAL FIELD

The present disclosure relates to stable pharmaceutical formulations that can reduce solid state reactions of different components within the pharmaceutical formulations.

### BACKGROUND

The chemical stability of drugs is one of their most critical quality attributes as the safety and the efficacy of the drug must be guaranteed throughout the shelf-life of the product. When incompatible solid materials are mixed in a pharmaceutical formulation, they may cause unwanted solid state reactions, leading to potential degradation of the active pharmaceutical ingredients (API). Solid-state reactions of drug substances can include oxidation, cyclization, hydrolysis, and deamidation.

Incompatible solid materials can include active pharmaceutical ingredients (API) and excipients. Two APIs can be incompatible with each other, causing unwanted solid state reactions and degradation of the API. An API can also be incompatible with one or more excipients, causing unwanted solid state reactions and degradation of the API. The solid state reactions and the degradation of the API may decrease the effectiveness of the formulation or increase the toxicity of the formulation.

Thus, there is a need for methods to develop stable pharmaceutical formulations to reduce solid state reactions and improve the stability of drug substances.

### SUMMARY

This disclosure is related to stable pharmaceutical formulations with reduced solid-state reactions. When incompatible solid materials are mixed in a pharmaceutical formulation, they may react or may aid the degradation of the drug. Without being bound by theory, the stable formulations described herein are based on the observation that inorganic oxide coatings on ingredient particles and/or drug particles can act as barriers, suppress unwanted solid state interactions, and improve the stability of the formulation.

In one aspect, the stable formulations comprise a coated ingredient particle and an optionally coated drug particle. The coating can be applied by vapor phase deposition. The coated particles have reduced solid state reactions compared to the uncoated particles, leading to improved stability. The ingredient can be a pharmaceutically acceptable excipient. The ingredient can be hydroscopic. In some embodiments, the ingredient can be CaCO₃ (calcium carbonate) or Mg(OH)₂ (magnesium hydroxide). The drug can be an organic molecule. In some embodiments, the drug can be famotidine.

In some embodiments, the drug can be aspirin and the ingredient can be magnesium stearate. In some embodiments, the drug can be famotidine and the ingredient can be magnesium hydroxide. In some embodiments, the drug can be a moisture sensitive drug and the ingredient can be magnesium hydroxide. In some embodiments, the drug can be a moisture sensitive drug and the ingredient can be polyvinylpyrrolidone (PVP). In some embodiments, the moisture sensitive drug can be selected from procaine, sulphonamides, chlorothiazide, barbituric acid, aspirin, alkaloids, penicillin, acyclovir, erythromycin base, itraconazole, and ketoconazole.

In some embodiment, the ingredient can be a second drug. In some embodiments, the drug can be aspirin and the ingredient can be acetaminophen. In some embodiments the drug can be codeine and the ingredient can be acetaminophen. In some embodiments, the drug can be clavulanic acid and the ingredient can be amoxicillin. In some embodiments, the drug can be cyclosporine and the ingredient can be rifampin. In some embodiments, the drug can be a penicillin derivative and the ingredient can be probenecid. In some embodiments, the drug can be a bronchodilator and the ingredient can be a corticosteroid. In some embodiments, the drug can be fluconazole and the ingredient can be bergapten.

In some embodiment, the ingredient can be a second drug. In some embodiments, the ingredient can be aspirin and the drug can be acetaminophen. In some embodiments the ingredient can be codeine and the drug can be acetaminophen. In some embodiments, the ingredient can be clavulanic acid and the drug can be amoxicillin. In some embodiments, the ingredient can be cyclosporine and the drug can be rifampin. In some embodiments, the ingredient can be a penicillin derivative and the drug can be probenecid. In some embodiments, the ingredient can be a bronchodilator and the drug can be a corticosteroid. In some embodiments, the ingredient can be fluconazole and the drug can be bergapten. The coating (e.g., aluminum oxide coating) described herein can act as a physical barrier and reduce interaction between the drug (e.g., famotidine) particles and the coated ingredient (e.g., CaCO₃ or Mg(OH)₂) particles in the formulation. The coating can also act as a physical barrier to reduce solid state reactions between a first ingredient (e.g., CaCO₃) particle and a second ingredient (e.g., Mg(OH)₂) particle. The coating can also act a physical barrier to reduce exposure to water/moisture by the coated drug particles and/or the coated ingredient particles. The coating can also act as a physical barrier to reduce exposure to oxygen/oxidants by the coated drug particles and/or the coated ingredient particles. The coating can reduce hydrolyzation and/or oxidation of the coated drug particles and/or the coated ingredient particles. The coating described herein may act as a physical barrier to reduce exposure of a hygroscopic ingredient (e.g., Mg(OH)₂) in the formulation to the environment.

An uncoated ingredient particle is coated with an ingredient coating layer to create the coated ingredient particle. In some embodiments, the stable formulation comprises a coated ingredient particle and an uncoated drug particle.

In some embodiments, an uncoated ingredient particle is coated with an ingredient coating layer to create the coated ingredient particle, and an uncoated drug particle is coated with a drug coating layer to create the coated drug particle. In some embodiments, the stable formulation comprises a coated ingredient particle and a coated drug particle. The ingredient coating layer of the coated ingredient particles can be the same as the drug coating layer on the coated drug particles.

Both the ingredient coating layer and the drug coating layer can be multi-layer coatings that include two or more inorganic oxide (e.g., metal oxide or metalloid oxide) layers. For example, both the ingredient coating layer and the drug coating layer can include an inner zinc oxide coating layer and an outer aluminum oxide coating layer. Both the ingredient coating layer and the drug coating layer can include a ternary compound coating layer, for example, an aluminum-zinc-oxide (AZO) coating. In the case where both the ingredient coating layer and the drug coating layer include an AZO coating, the Al/Zn ratio of the ingredient coating layer can be different from the Al/Zn ratio of the drug coating layer.

In one aspect, the disclosure is related to an pharmaceutical composition, comprising:
(a) a coated ingredient particle consisting of an ingredient-containing core comprising an ingredient, and an ingredient coating layer enclosing the ingredient-containing core; and
(b) an optionally coated drug particle,
wherein the ingredient coating layer comprises an inorganic oxide that comprises aluminum, zinc, silicon, and/or titanium.

In some embodiments, the drug particle is a coated drug particle that comprises a drug-containing core comprising a drug and a drug coating layer enclosing the drug-containing core, wherein the drug coating layer comprises an inorganic oxide that comprises aluminum, zinc, silicon, and/or titanium.

In some embodiments, the ingredient coating layer in the coated ingredient particle is the same as the drug coating layer in the coated drug particle.

In some embodiments, the ingredient coating layer is 0.1 nm-120 nm thick.

In some embodiments, the ingredient coating layer is 5 nm-15 nm thick.

In some embodiments, the coated ingredient particle comprises 1-20% wt/wt inorganic oxide.

In some embodiments, the ingredient-containing core consists of an ingredient.

In some embodiments, the ingredient-containing core comprises an ingredient and one or more pharmaceutically acceptable excipients.

In some embodiments, the ingredient-containing core has a D50 on a volume average basis of 100 nm-30 micrometers.

In some embodiments, the ingredient-containing core has a median particle size, on a volume average basis, between 0.1 μm and 20 μm.

In some embodiments, the drug is an organic molecule.

In some embodiments, the drug is famotidine.

In some embodiments, the ingredient is a pharmaceutically acceptable excipient or a second drug that is different from the drug in the drug particle.

In some embodiments, the ingredient and the drug can undergo a solid state reaction.

In some embodiments, the ingredient is hygroscopic.

In some embodiments, the ingredient is calcium carbonate.

In some embodiments, the ingredient is magnesium hydroxide.

In some embodiments, the composition comprises a dry mix of the coated ingredient particle and the drug particle.

In some embodiments, the composition further comprises a pharmaceutically acceptable excipient or carrier.

In some embodiments, the ingredient coating layer stabilizes the formulation, leading to less impurities after 1 month of storage at 40° C. and 75% relative humidity.

In some embodiments, the ingredient coating layer slows the release rate of the coated ingredient particle.

In some embodiments, ingredient coating layer increases the flowability of the coated ingredient particle.

In one aspect, the disclosure is related to A method of preparing a pharmaceutical composition, the method comprising the sequential steps of:
(a) loading an ingredient-containing core comprising an ingredient into a chamber of a reactor;
(b1) applying a vaporous or gaseous precursor to the particles in the reactor by pulsing the vaporous or gaseous aluminum precursor into the reactor;
(b2) purging using an inert gas;
(b3) applying a vaporous or gaseous oxidant to the particles in the reactor by pulsing the oxidant into the reactor;
(b4) purging using an inert gas;
(c) repeating steps (b1)-(b4) at least once to create a coated ingredient particle; and
(d) mixing the coated ingredient particle with an optionally coated drug particle.

In some embodiments, each purging step comprises flowing the inert gas into the reactor chamber to reach a desired pressure and after a delay time pumping the inert gas out of the reactor until the pressure of the inert gas is below 10 torr.

In some embodiments, the drug particle is a coated drug particle that is prepared by a method comprising the sequential steps of:
(a) loading a drug-containing core comprising a drug into a chamber of a reactor;
(b1) applying a vaporous or gaseous precursor to the particles in the reactor by pulsing the vaporous or gaseous aluminum precursor into the reactor;
(b2) purging using an inert gas;
(b3) applying a vaporous or gaseous oxidant to the particles in the reactor by pulsing the oxidant into the reactor;
(b4) purging using an inert gas;
(c) repeating steps (b1)-(b4) at least once to create the coated drug particle.

In some embodiments, the resulting pharmaceutical composition comprises a coated ingredient particle consisting of an ingredient-containing core comprising an ingredient, and an ingredient coating layer enclosing the ingredient-containing core, wherein the ingredient coating layer is conformal, pinhole-free and comprises an inorganic oxide that comprises aluminum, zinc, silicon, and/or titanium.

In some embodiments, the resulting pharmaceutical composition comprises (a) a coated ingredient particle consisting of an ingredient-containing core comprising an ingredient, and an ingredient coating layer enclosing the ingredient-containing core; and (b) a coated drug particle consisting of a drug-containing core comprising a drug, and a drug coating layer enclosing the drug-containing core.

In some embodiments, the drug particle is a coated drug particle that comprises a drug-containing core comprising a drug, and a drug coating layer enclosing the drug-containing core, wherein the drug coating layer is conformal, pinhole-free and comprises an inorganic oxide that comprises aluminum, zinc, silicon, and/or titanium.

In some embodiments, steps (b1)-(b4) take place at a temperature between 25° C. and 60° C.

In some embodiments, step (a) further comprises agitating the ingredient-containing particle.

In some embodiments, the precursor is an aluminum oxide precursor.

In some embodiments, the precursor is trimethylaluminium (TMA).

In some embodiments, the oxidant is water.

In some embodiments, the coating constitutes 1-20% wt/wt of the coated particles.

In some embodiments, the ingredient coating layer has a thickness in the range of 0.1 nm to 120 nm.

In some embodiments, the ingredient coating layer has a thickness in the range of 5 nm to 15 nm.

In some embodiments, the ingredient is not degraded during the coating process.

In one aspect, the disclosure is related to a method of preparing a pharmaceutical composition, the method comprising the sequential steps of:
(a) loading an ingredient-containing core comprising an ingredient into a chamber of a reactor;
(b) performing a first number of first cycles, wherein each first cycle comprises steps (b1)-(b4):
   (b1) applying a vaporous or gaseous first inorganic oxide precursor to the particles in the reactor by pulsing the vaporous or gaseous first inorganic oxide precursor into the reactor;
   (b2) purging using an inert gas;
   (b3) applying a vaporous or gaseous oxidant to the particles in the reactor by pulsing the oxidant into the reactor;
   (b4) purging using an inert gas;
(c) performing a second number of second cycles, wherein each second cycle comprises steps (c1)-(c4):
   (c1) applying a vaporous or gaseous second inorganic oxide precursor to the particles in the reactor by pulsing the vaporous or gaseous second inorganic oxide precursor into the reactor;
   (c2) purging using an inert gas;
   (c3) applying a vaporous or gaseous oxidant to the particles in the reactor by pulsing the oxidant into the reactor;
   (c4) purging using an inert gas; and
(d) repeating steps (b)-(c) at least once to create a coated ingredient particle; and
(e) mixing the coated ingredient particle with an optionally coated drug particle,
wherein, the first inorganic oxide precursor and second inorganic oxide precursor are different and the first number is an integer selected from 1-10, and the second number is an integer selected from 1-10.

In some embodiments, the uncoated ingredient particles have a median particle size, on a volume average basis between 0.1 μm and 1000 μm.

In some embodiments, the drug particle is a coated drug particle prepared by a method comprising the sequential steps of:
(a) loading a drug-containing core comprising a drug into a chamber of a reactor;
(b) performing a first number of first cycles, wherein each first cycle comprises steps (b1)-(b4):
   (b1) applying a vaporous or gaseous first inorganic oxide precursor to the particles in the reactor by pulsing the vaporous or gaseous first inorganic oxide precursor into the reactor;
   (b2) purging using an inert gas;
   (b3) applying a vaporous or gaseous oxidant to the particles in the reactor by pulsing the oxidant into the reactor;
   (b4) purging using an inert gas;
(c) performing a second number of second cycles, wherein each second cycle comprises steps (c1)-(c4):
   (c1) applying a vaporous or gaseous second inorganic oxide precursor to the particles in the reactor by pulsing the vaporous or gaseous second inorganic oxide precursor into the reactor;
   (c2) purging using an inert gas;
   (c3) applying a vaporous or gaseous oxidant to the particles in the reactor by pulsing the oxidant into the reactor;
   (c4) purging using an inert gas; and
(d) repeating steps (b)-(c) at least once to create a coated drug particle,
wherein, the first inorganic oxide precursor and second inorganic oxide precursor are different and the first number is an integer selected from 1-10, and the second number is an integer selected from 1-10.

In some embodiments, the resulting pharmaceutical composition comprises a coated ingredient particle consisting of an ingredient-containing core comprising an ingredient, and an ingredient coating layer enclosing the ingredient-containing core, wherein the ingredient coating layer is conformal, pinhole-free and comprises an inorganic oxide that comprises aluminum, zinc, silicon, and/or titanium.

In some embodiments, the resulting pharmaceutical composition comprises (a) a coated ingredient particle consisting of an ingredient-containing core comprising an ingredient, and an ingredient coating layer enclosing the ingredient-containing core; and (b) a coated drug particle consisting of a drug-containing core comprising a drug, and a drug coating layer enclosing the drug-containing core.

In some embodiments, the drug particle is a coated drug particle that comprises a drug-containing core comprising a drug, and a drug coating layer enclosing the drug-containing core, wherein the drug coating layer is conformal, pinhole-free and comprises an inorganic oxide that comprises aluminum, zinc, silicon, and/or titanium.

In some embodiments, the first inorganic oxide precursor is an aluminum oxide precursor and the second inorganic oxide precursor is a zinc oxide precursor.

In some embodiments, the first inorganic oxide precursor is a zinc precursor and the second inorganic oxide precursor is an aluminum oxide precursor.

In some embodiments, the aluminum oxide precursor is trimethylaluminum (TMA).

In some embodiments, the zinc oxide precursor is diethylzinc (DEZ).

In some embodiments, the coating constitutes 1-20% wt/wt of the coated particles.

In some embodiments, each of the first and second inorganic oxide precursor is selected from DEZ and TMA and either: a) the first inorganic oxide precursor is TMA and the second inorganic oxide precursor is DEZ; or b) the first inorganic oxide precursor is DEZ and the second inorganic oxide precursor is TMA.

In some embodiments, the first number is 1 or 2 and the second number is an integer between 1 and 10.

In some embodiments, the first number is 1.

In some embodiments, the second number is 2, 3, 4 or 5.

In some embodiments, the second number is 3.

In some embodiments, the second number is 4.

In some embodiments, steps (b)-(c) occur 1-40 times.

In some embodiments, each of steps (b1), (b3), (c1) and (c3) comprises: (i) introducing the vaporous or gaseous inorganic oxide precursor into the chamber, (ii) allowing a holding time to pass, and (iii) pumping the vaporous or gaseous inorganic oxide precursor of the chamber; and repeating steps (i)-(ii) at least once.

In some embodiments, some or all of the residual vaporous or gaseous first inorganic oxide precursor is pumped out of the reactor prior to step (b3).

In some embodiments, some or all of the residual vaporous or gaseous oxidant is pumped out of the reactor prior to step (c).

In some embodiments, some or all of the residual vaporous or gaseous second inorganic oxide precursor is pumped out of the reactor prior to step (c3).

In some embodiments, the first cycles and second cycles take place at a temperature between 25° C. and 60° C.

In some embodiments, the oxidant in step (b3) is water.

In some embodiments, the oxidant in step (c3) is water.

In some embodiments, step (a) further comprises agitating the particles.

In some embodiments, each purging step comprises flowing the inert gas into the reactor chamber to a desired pressure and after a delay time pumping the inert gas out of the reactor until the pressure of the inert gas is below 10 torr and repeating the steps of flowing the inert gas into the reactor chamber to a desired pressure and after a delay time pumping the inert gas out of the reactor until the pressure of the inert gas is below 10 torr.

In some embodiments, the inorganic oxide coating is a ternary compound.

In some embodiments, the ternary compound is composed of aluminum, zinc and oxygen.

In some embodiments, the method further comprises agitating the particles in the reactor throughout steps (a)-(d).

In some embodiments, the particles are not removed from the reactor during steps (a)-(d).

In some embodiments, the ingredient coating layer has a thickness in the range of 0.1 nm to 120 nm.

In some embodiments, the ingredient coating layer has a thickness in the range of 5 nm to 15 nm.

In some embodiments, the ingredient is not degraded during the coating process.

In one aspect, the disclosure is related to a composition prepared by the method described herein.

The coating process can be performed at a low process temperature, e.g., below 80° C., e.g., at or below 50° C., at or below 35° C., or at or below 25° C. In some embodiments, the operating temperature is 50° C. In some embodiments, the operating temperature is above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. (e.g. 20° C. to 80° C.). In some embodiments, the operating temperature is below 20° C., below 25° C., below 30° C., below 35° C., below 40° C., below 45° C., below 50° C., below 56° C., below 60° C., below 65° C., below 70° C., below 75° C., or below 80° C. In particular, the particles can remain or be maintained at such temperatures during all of the coating steps (e.g., inorganic precursor steps, oxidant steps, pump-purge steps). This can be achieved by having the oxidant gas, precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

In some embodiments, the amount of inorganic component constitutes more than 0.1%, more than 0.2%, more than 0.3%, more than 0.4%, more than 0.5%, more than 0.6%, more than 0.7%, more than 0.8%, more than 0.9%, more than 1%, more than 1.2%, more than 1.4%, more than 1.6%, more than 1.8%, more than 2%, more than 2.2%, more than 2.4%, more than 2.6%, more than 2.8%, more than 3%, more than 3.2%, more than 3.4%, more than 3.6%, more than 3.8%, more than 4%, more than 4.2%, more than 4.4%, more than 4.6%, more than 4.8%, more than 5%, more than 6%, more than 7%, more than 8%, more than 9%, more than 10%, more than 12%, more than 14%, more than 16%, more than 18%, or more than 20% wt/wt of the coated particles.

In some embodiments, weight percent of inorganic oxides in the coated particles is less than 0.1%, less than 0.2%, less than 0.3%, less than 0.4%, less than 0.5%, less than 0.6%, less than 0.7%, less than 0.8%, less than 0.9%, less than 1%, less than 1.2%, less than 1.4%, less than 1.6%, less than 1.8%, less than 2%, less than 2.2%, less than 2.4%, less than 2.6%, less than 2.8%, less than 3%, less than 3.2%, less than 3.4%, less than 3.6%, less than 3.8%, less than 4%, less than 4.2%, less than 4.4%, less than 4.6%, less than 4.8%, less than 5%, less than 6%, less than 7%, less than 8%, less than 9%, less than 10%, less than 12%, less than 14%, less than 16%, less than 18%, or less than 20% wt/wt of the coated particles. In some embodiments, the weight percent of inorganic oxides in the coated particles is 0.1%-20%, 0.5%-10%, 1%-10%, 1%-5%, 2%-5%, 1%-4%, 1%-3%, or 2%-4% wt/wt of the coated particles. In some embodiments, the amount of inorganic component constitutes about 1%-20% wt/wt of the coated particles.

In some embodiments, the coated particles exhibit increased hydrophobicity comparing to the uncoated particles.

In some embodiments, the coated particles exhibit increased powder flowability (“FFc”) comparing to the uncoated particles.

In some embodiments, the coated particles exhibit increased bulk density comparing to the uncoated particles.

In one aspect, the disclosure relates to a method to coat particles using supercycles. A supercycle includes a first number of first cycles (e.g., TMA cycles) and a second number of second cycles (e.g., DEZ cycles). In some embodiments, the aluminum/zinc (Al/Zn) ratio in the coating can be adjusted by varying the number of first cycles (e.g., TMA cycles) and the number of second cycles (e.g., DEZ cycles). In some embodiments, the first number is an integer selected from 1-10, and the second number is an integer selected from 1-10. In some embodiments, the first number is 1, 2, 3, 4, 5, 6, 7, 8, 9, 10, 11, 12, 13, 14, 15, 16, 17, 18, 19, 20, 21, 22, 23, 24, 25, 30, 40, or 50. In some embodiments, the second number is 1, 2, 3, 4, 5, 6, 7, 8, 9, 10, 11, 12, 13, 14, 15, 16, 17, 18, 19, 20, 21, 22, 23, 24, 25, 30, 40, or 50. In some embodiments, the first number is 1 and the second number is an integer selected from 1-5. In some embodiments, the first number is 1 and the second number is 2. In some embodiments, the first number is 1 and the second number is 3. In some embodiments, the first number is 1 and the second number is 4.

In some embodiments, multiple supercycles are used to create a mixed (e.g., ternary compound) coating layer (AZO coating layer or ASO coating layer). In some embodiments, the number of supercycles is more than 1, more than 2, more than 3, more than 4, more than 5, more than 6, more than 7, more than 8, more than 9, more than 10, more than 15, more than 20, more than 25, more than 30, more than 35, more than 40, more than 45, more than 50, more than 60, more than 70, more than 80, more than 90, more than 100, more than 110, more than 120, more than 130, more than 140, or more than 150. In some embodiments, the number of supercycles is less than 2, less than 3, less than 4, less than 5, less than 6, less than 7, less than 8, less than 9, less than 10, less than 15, less than 20, less than 25, less than 30, less than 35, less than 40, less than 45, less than 50, less than 60, less than 70, less than 80, less than 90, less than 100, less than 110, less than 120, less than 130, less than 140, or less than 150. In some embodiments, the number of supercycles is 5-50, 5-25, or 5-10.

In some embodiments, multiple first supercycles with a first Al/Zn ratio are followed by multiple second supercycles with a second Al/Zn ratio to deposit an AZO coating with two or more different Al/Zn ratios or a gradient Al/Zn ratio.

In some embodiments, multiple supercycles are used to create a mixed aluminum and silicon oxide coating layer (ASO coating layer). In some embodiments, multiple first supercycles with a first Al/Si ratio are followed by multiple second supercycles with a second Al/Si ratio to deposit an ASO coating with two or more different Al/Si ratios or a gradient Al/Si ratio.

In some embodiments, 5-10 supercycles are performed wherein the first number is 1 and the second number is 3. In some embodiments, 5-25 additional supercycles are performed wherein the first number is 1 and the second number is 2.

In some embodiments, 5-10 additional supercycles are performed wherein the first number is 1 and the second number is 4. In some embodiments, 5-25 additional supercycles are performed wherein the first number is 1 and the second number is 3.

In some embodiments there are multiple different layers. For example an AZO layer can be combined with AlOx layer and/or ZnOx layer to provide various different coating structures. The AZO layer can be an inner layer and the AlOx or ZnOx layer can be an outer layer. In some embodiments, exemplary coating structures include an AlOx layer and an AZO layer or an ZnOx layer and an AZO layer.

In some embodiments, the coated particles contain a mixture of different coating layers. For example, an ASO layer can be combined with AlOx layer and/or SiOx layer to provide various different coating structures. In some embodiments, the ASO layer can be an inner layer and the AlOx or SiOx layer can be an outer layer. In some embodiments, exemplary coating structures include an AlOx layer and an ASO layer or an SiOx layer and an ASO layer.

In some embodiments, the aluminum/zinc (Al/Zn) ratio (wt/wt) in the coated particles is more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25, more than 0.3, more than 0.35, more than 0.4, more than 0.45, more than 0.5, more than 0.6, more than 0.7, more than 0.9, or more than 0.9. In some embodiments, the aluminum/zinc (Al/Zn) ratio in the coated particles is less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. In some embodiments, the Al/Zn ratio in the coated particles is 0.1-0.5, 0.1-0.4, 0.1-0.3, 0.15-0.3 or 0.15-0.35.

In some embodiments, the entirety of the coating has a thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the entirety of the coating has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the entirety of the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the entirety of the coating has a thickness of between 10 nm and 50 nm. In some embodiments, the entirety of the coating has a thickness of between 10 nm and 200 nm, between 10 nm and 100 nm, between 10 nm and 50 nm, or between 25 nm and 50 nm. In some embodiments, the entirety of the coating has a thickness of 10-60 nm, 10-50 nm, 10-40 nm or 10-30 nm.

In some embodiments, an individual layer in a multi-layer coating has thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the aluminum oxide layer has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the coating has a thickness of between 1 nm and 30 nm. In some embodiments, the coating has a thickness of between 1 nm and 20 nm. In some embodiments, the coating has a thickness of 2-5 nm, 5-10 nm, or 10-20 nm. In some embodiments, individual AZO, ASO, ZnOx, AlOx or SiOx layers can have a thickness of 1-10 nm, 5-10 nm, 5-20 nm or 10-20 nm.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a composition, “about” may mean+/−10% of the recited value. For instance, a composition including about 100 ng/ml of a given compound may include 90˜110 ng/ml of the compound.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 depicts a schematic illustration of an exemplary reactor system.

FIG. 2 depicts a schematic illustration of an exemplary stable formulation comprising a coated ingredient (antacid) particle and a coated drug (API) particle.

FIG. 3 depicts a schematic illustration of an exemplary supercycle. The exemplary supercycle contains n number of AlOx cycles and m number of ZnOx cycles. The AlOx cycle includes applying a vaporous or gaseous aluminum precursor (e.g., trimethylaluminum or TMA); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas. The ZnOx cycle includes applying a vaporous or gaseous zinc precursor (e.g., diethylzinc or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 4 depicts a schematic illustration of an exemplary cycle in a supercycle. The cycle contains applying a vaporous or gaseous precursor (e.g., TMA or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 5 depicts a schematic illustration of particle coated with multiple layers. In this example, an AlOx layer is applied using conventional cycles, then an AZO layer is applied using supercycles. Finally a ZnOx layer is applied using conventional cycles. The Al/Zn ratio in the AZO layer can be adjusted by varying the numbers of AlOx cycles and ZnOx cycles. The overall coating thickness and the coating wt % can be adjusted by varying the number of conventional cycles and the number of supercycles.

FIGS. 6A-6B depict the morphology of the uncoated drug particles and ingredient particles before coating. FIG. 6A shows magnesium hydroxide. FIG. 6B shows calcium carbonate. FIG. 6C shows famotidine.

FIG. 7 shows the flowability testing results of the coated and uncoated ingredient particles.

FIGS. 8A-8C show the elemental composition of the uncoated drug particles and ingredient particles before coating. FIG. 8A shows magnesium hydroxide particles. FIG. 8B shows calcium carbonate particles. FIG. 8C shows famotidine particles.

FIG. 9 shows a flow chart for an exemplary impurity analysis method.

FIG. 10 shows the results of impurity analysis for coated and uncoated drug and ingredient particles.

FIG. 11 shows the results of impurity analysis for coated and uncoated drug and ingredient particles. The coated API tablets were prepared by pressing together coated drug (famotidine) particles and coated ingredient particles (magnesium hydroxide and calcium carbonate).

FIG. 12 shows the possible challenges in preparing a stable formulation that comprises famotidine, calcium carbonate, and magnesium hydroxide.

FIG. 13 shows the results of the dissolution of uncoated and coated famotidine drug particles.

### DETAILED DESCRIPTION

This disclosure pertains to methods for preparing a stable formulation that comprises an optionally coated drug particles and coated ingredient particles comprising an ingredient-containing core and a coating comprising at least one inorganic oxide. The coating can be applied by vapor phase deposition.

### Solid State Reactions

Solid-state reactions of drug substances can include oxidation, cyclization, hydrolysis, and deamidation.

Solid-state reactions of the drug substance can occur in cases where the drug substance is intrinsically chemically reactive or unstable. In such cases, the formulation can accelerate degradation in any or all of the following ways: (1) acceleration due to interaction with excipients; (2) acceleration due to processing effects; (3) acceleration induced by excipients (but not involving chemical reactions with the excipient).

Often, acceleration of reaction is due to the creation or presence of amorphous material, which has an enhanced chemical reactivity. In such cases processing or, possibly, simply interaction with the excipients can increase the amount of amorphous drug substance. This amorphous drug substance will then react due to its increased mobility and ability to interact with moisture.

Direct reaction of excipients with the drug substance can also occur, such as solid-solid acid base reactions. However, buffers or acids and bases are used to stabilize drug substances in formulations as well. In many cases these effects can be determined by mixing the drug substance with various excipients. In other cases processing is needed to induce the reaction.

In general, the susceptibility of an API towards reactions with excipients is dependent on the existence of a possible chemical reaction pathway and upon the energy of the associated transition state, which acts as an energy barrier for the reaction.

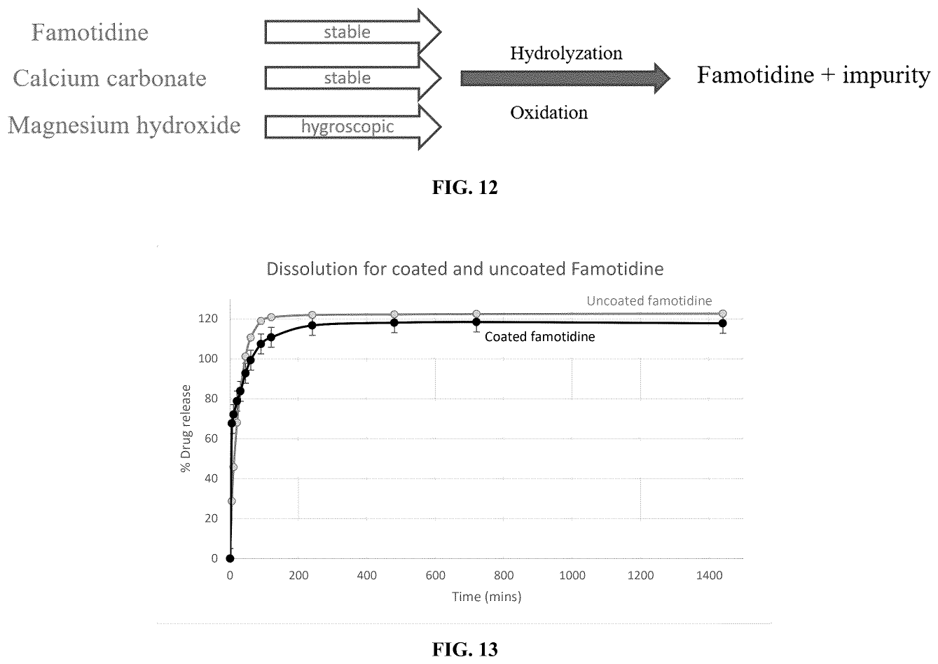

As shown in FIG. 12, the possible challenges in preparing a stable famotidine formulation that comprises famotidine, calcium carbonate, and magnesium hydroxide include hydrolyzation and oxidation. Without being bound by theory, the hygroscopic magnesium hydroxide in the famotidine formulation may attract water/moisture from the environment to accelerate the degradation of the drug (famotidine). The coating described herein may act as a physical barrier to reduce exposure of the drug to water/moisture. The coating described herein may act as a physical barrier to reduce exposure of the hygroscopic magnesium hydroxide to the environment.

### Drug

The drug (therapeutic agent or API) can be any therapeutic agent used for the treatment of a condition or disease, a pharmaceutically acceptable salt thereof, or an analogue of either of the foregoing. The drug can be an organic molecule. For example, the drug can be famotidine.

### Ingredient

The current disclosure relates to a stable formulation that comprises (1) an optionally coated drug particle and (2) a coated ingredient particle. The ingredient can be either a second drug in the formulation, or a non-drug ingredient (e.g., excipient) in the formulation. The ingredient may be a pharmaceutically acceptable excipient. The ingredient may be hygroscopic. The ingredient may be an inorganic molecule. For example, the ingredient may be Mg(OH)₂ (magnesium hydroxide) and/or CaCO₃ (calcium carbonate).

### Uncoated Particles (Starting Materials)

The uncoated particles may be ingredient particles that will be coated by the methods described herein. The uncoated particles may be drug particles that will be coated by the methods described herein. The uncoated particles may further contain one or more pharmaceutically acceptable excipients.

The uncoated particles may contain at least 10%, 20%, 30%, 40%, or 50% wt/wt ingredients. The uncoated particles may contain less than 10%, 20%, 30%, 40%, or 50% wt/wt ingredients. The uncoated particles may contain at least 60%, 70%, 80%, 90%, 99% or 100% wt/wt ingredients. The uncoated particles may contain 10-20%, 10-40%, 10-60%, 20-70%, 30-80%, 50%-90%, or 70-100% wt/wt/ingredients.

The uncoated particles may contain at least 10%, 20%, 30%, 40%, or 50% wt/wt drugs. The uncoated particles may contain less than 10%, 20%, 30%, 40%, or 50% wt/wt drugs. The uncoated particles may contain at least 60%, 70%, 80%, 90%, 99% or 100% wt/wt drugs. The uncoated particles may contain 10-20%, 10-40%, 10-60%, 20-70%, 30-80%, 50%-90%, or 70-100% wt/wt/drugs.

The uncoated particles may have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D10 of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D10 of 0.1 μm to 200 μm, 0.1 μm to 1 μm, 0.1 μm to 10 μm, or 0.1 μm to 50 μm on a volume average basis. The uncoated particles may have a D10 of about 2 μm on a volume average basis.

The uncoated particles may have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D50 of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D50 of 0.1 μm to 200 μm, 0.1 μm to 1 μm, 0.1 μm to 10 μm, or 0.1 μm to 50 μm on a volume average basis. The uncoated particles may have a D50 of about 4.5 μm on a volume average basis.

The uncoated particles may have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D90 of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D90 of 200 μm to 2000 μm on a volume average basis. The uncoated particles may have a D50 of 0.1 μm to 200 μm, 0.1 μm to 1 μm, 0.1 μm to 10 μm, or 0.1 μm to 50 μm on a volume average basis. The uncoated particles may have a D90 of about 9.2 μm on a volume average basis.

### Vapor Phase Deposition

Coatings can be applied to the uncoated particles by vapor phase deposition using a precursor molecule (e.g., an inorganic oxide precursor) and an oxidant (e.g., ozone or water vapor). Vapor phase deposition of inorganic oxides (e.g., metal oxides or metalloid oxides) is sometimes referred to as atomic layer deposition (ALD). However, depending on a number of factors, including the surface being coated, each cycle of the deposition reaction does not necessarily deposit one atomic layer on the entire surface.

### Reactor System

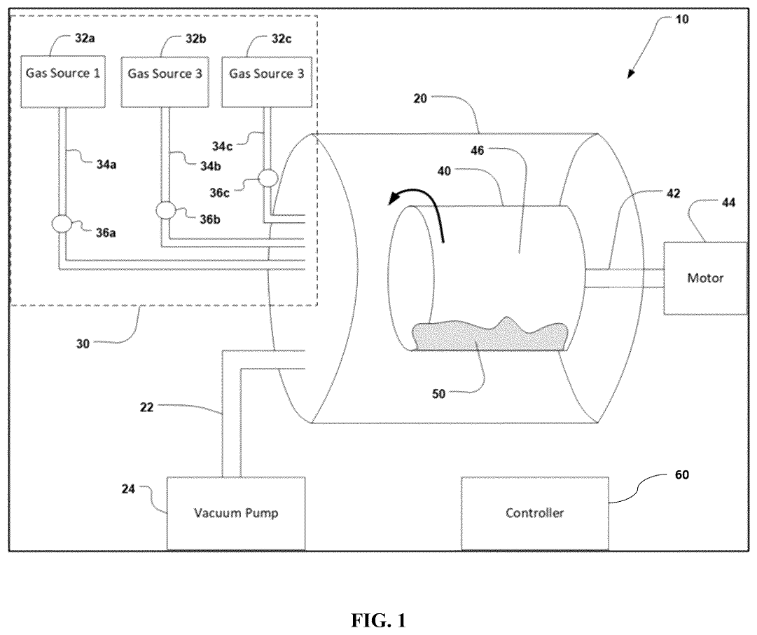

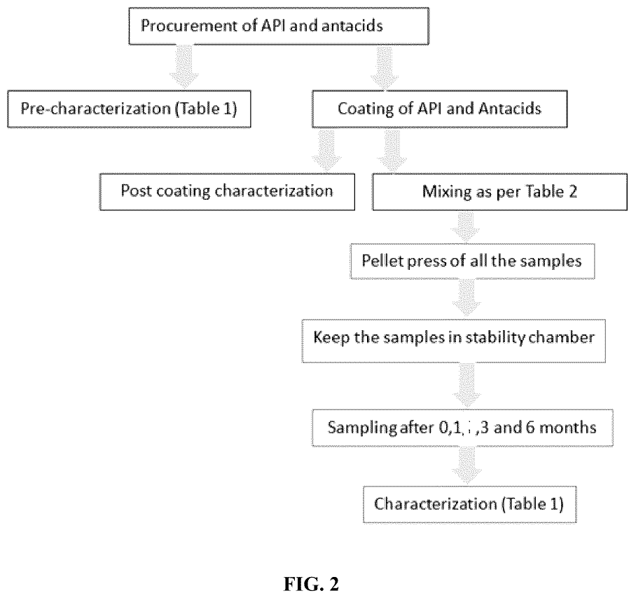

The term “reactor system” in its broadest sense includes all systems that could be used to perform vapor phase deposition or atomic layer deposition. An exemplary reactor system is illustrated in FIG. 1 and further described below.

The reactor system 10 can perform vapor phase deposition or atomic layer deposition. The reactor system 10 permits the process to be performed at higher (above 50° C., e.g., 50-100° C. or higher) or lower process temperature, e.g., below 50° C., e.g., at or below 25° C. For example, the reactor system 10 can form thin-film inorganic oxides on the particles primarily at temperatures of 40-80° C., e.g., 40° C. or 80° C. In general, the particles can remain or be maintained at such temperatures. This can be achieved by having the reactants and/or the interior surfaces of the reactor chamber (e.g., the chamber 20 and drum 40 discussed below) remain or be maintained at such temperatures.

Again, illustrating a vapor phase deposition or atomic layer deposition process, the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the vapor phase deposition or atomic layer deposition process by introducing a gaseous oxidant and aluminum (or zinc) precursor into the chamber 20. The gaseous oxidant and aluminum (or zinc) precursor are introduced alternatively into the reactor. In addition, the reaction can be performed at low temperature conditions, such as below 80° C., e.g., below 50° C., below 30° C., or below 25° C. The operating temperature may be 50° C. The operating temperature may be above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. The operating temperature may be below 20° C., below 25° C., below 30° C., below 35° C., below 40° C., below 45° C., below 50° C., below 56° C., below 60° C., below 65° C., below 70° C., below 75° C., or below 80° C.

The chamber 20 is also coupled to a chemical delivery system 30. The chemical delivery system 30 includes three or more gas sources 32a, 32b, 32c coupled by respective delivery lines 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 1 illustrates separate gas lines extending in parallel to the chamber for each gas source, two or more of the gas lines could be joined, e.g., by one or more three-way valves, before the combined line reaches the chamber 20.

One of the gas sources can provide an oxidant. In particular, a gas source can provide a vaporous or gaseous oxidant. For example, the oxidant can be ozone. As another example, the oxidant can be water vapor.

One of the gas sources can be an aluminum (or zinc) precursor. In particular, a gas source can provide a vaporous or gaseous aluminum (or zinc) precursor. For example, the aluminum precursor can be TMA.

One of the gas sources can provide a purge gas. In particular, the third gas source can provide a gas that is chemically inert to the oxidant and aluminum (or zinc) precursor, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 1-10 μm.

In operation, one of the gasses flows into chamber 20 from the chemical delivery system 30 as the drum 40 rotates. A combination of pores (1-100 μm), holes (0.1-10 mm), or large openings in the coating drum 40 serve to confine the particles in the coating drum 40 while allowing rapid delivery of precursor chemistry and the pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to expose new surfaces of the powder bed, ensuring a large surface area of the particles remains exposed to the process gas. This permits fast, uniform interaction of the particle surface with the process gas.

In some implementations, one or more temperature control components are integrated into the drum 40 to permit control of the temperature of the drum 40. For example, a resistive heater, a thermoelectric cooler, or other component can be in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44, etc. The controller 60 can receive the recipe as computer-readable data (e.g., that is stored on a non-transitory computer readable medium).

The controller 60 and other computing device parts of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, uncoated particles (e.g., drug particles and/or ingredient particles) are loaded into the drum 40 in the reactor system 10. Once any access ports are sealed, the controller 60 operates the reactor system 10 according to the recipe in order to form the thin-film inorganic oxides on the particles.

In one example, the uncoated particles are coated with an inorganic oxide coating. The uncoated particles can be coated with two reactant gases. The uncoated particles can be coated with a first reactant gas in the form of an inorganic precursor gas and a second reactant gas in the form of an oxidant gas. The inorganic oxide may be aluminum oxide, zinc oxide, titanium oxide, or silicon oxide. The inorganic oxide may be aluminum oxide. The inorganic precursor may be trimethylaluminium (TMA). The inorganic oxide may be zinc oxide. The inorganic precursor may be diethylzinc. The inorganic oxide may be titanium oxide. The inorganic precursor may be titanium tetrachloride (TiCl4). The inorganic oxide may be silicon oxide. The inorganic precursor may be 1,2-Bis(diisopropylamino)disilane (BDIPADS) or silicon tetrachloride. The oxidant may be water or ozone.

In particular, the two reactant gases are alternately supplied to the chamber 20, with each step of supplying a reactant gas followed by a purge cycle in which the inert gas is supplied to the chamber 20 to force out the reactant gas and by-products used in the prior step. Moreover, one or more of the gases (e.g., the reactant gases and/or the inert gas) can be supplied in pulses in which the chamber 20 is filled with the gas to a specified pressure, a delay time is permitted to pass, and the chamber is evacuated by the vacuum pump 24 before the next pulse commences.

In particular, the controller 60 can operate the reactor system 10 as follows.

In a first reactant cycle (called a half-cycle), while the motor 44 rotates the drum 40 to agitate the particles 50:
i) The gas distribution system 30 is operated to flow the first reactant gas, e.g., TMA, from the source 32a into the chamber 20 until a first specified pressure is achieved. The specified pressure can be 0.1 Torr to half of the saturation pressure of the reactant gas.
ii) Flow of the first reactant is halted, and a specified delay time is permitted to pass, e.g., as measured by a timer in the controller. This permits the first reactant to flow through the particle bed in the drum 40 and react with the surface of the particles 50 inside the drum 40.
iii) The vacuum pump 50 evacuates the chamber 20, e.g., down to pressures below 1 Torr, e.g., to 1 to 100 mTorr, e.g., 50 mTorr.

These steps (i)-(iii) can be repeated a number of times set by the recipe, e.g., two to ten times, e.g., six times.

Next, in a first purge cycle, while the motor 44 rotates the drum to agitate the particles 50:
iv) The gas distribution system 30 is operated to flow the inert gas, e.g., N₂, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the reactant gas and any vaporous by-products.
vi) The vacuum pump 50 evacuates the chamber 20, e.g., down to pressures below 10 Torr, e.g., to 1 to 500 mTorr, e.g., 50 mTorr.

These steps (iv)-(vi) can be repeated a number of times set by the recipe, e.g., six to twenty times, e.g., sixteen times. Taken together steps (iv)-(vi) are called a pump-purge cycle.

In a second reactant half-cycle, while the motor 44 rotates the drum 40 to agitate the particles 50:
vii) The gas distribution system 30 is operated to flow the second reactant gas, e.g., H2O, from the source 32b into the chamber 20 until a third specified pressure is achieved. The third pressure can be 0.1 Torr to half of the saturation pressure of the reactant gas.
viii) Flow of the second reactant is halted, and a specified delay time is permitted to pass, e.g., as measured by the timer in the controller. This permits the second reactant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

These steps (vii)-(ix) can be repeated a number of times set by the recipe, e.g., two to ten times, e.g., six times.

Next, a second purge cycle is performed. This second purge cycle can be identical to the first purge cycle, or can have a different number of repetitions of the steps (iv)-(vi) and/or different delay time and/or different pressure.

The cycle of the first reactant half-cycle, first purge cycle, second reactant half cycle and second purge cycle can be repeated a number of times set by the recipe, e.g., one to ten times.

The uncoated particles may be coated with one or more inorganic oxide coatings, for example, the uncoated particles may be coated with a combination of aluminum oxide, titanium oxide, zinc oxide and/or silicon oxide coatings. The uncoated particles may be first coated with an aluminum oxide layer and then coated with a silicon oxide layer. The uncoated particles may be first coated with an aluminum oxide layer, then coated with a silicon oxide layer, and then coated with an aluminum oxide layer.

Exemplary methods for applying aluminum oxide, titanium oxide, zinc oxide, silicon oxide coatings, multiple coating layers and supercycle coatings are provided below.

### Methods for Aluminum Oxide Coating

In one aspect, the disclosure provides methods for preparing stable formulations comprising uncoated particles (e.g., drug particles and/or ingredient particles) encapsulated by aluminum oxide coating.

The aluminum oxide coating can be performed on a rotary powder coating chamber. The operating temperature can be 35-50° C. or about 50° C. For each aluminum oxide coating process, one precursor may be introduced to reach a pressure of 0.3-2 torr for a hold time of 60 seconds, before nitrogen gas is used to purge the excess reactants and side products. The second precursor may then be introduced to reach a pressure of 2-8 torr for a hold time of 60 seconds, before nitrogen gas is used to purge the excess reactants and side products. This completes one coating cycle. Desired cycle numbers can be decided and aluminum oxide coating can be coated in repeated cycles to obtain a desired thickness.

The aluminum oxide coating can be applied using vapor phase deposition as described herein. The aluminum precursors can be trimethylaluminum (TMA). The oxidant can be water.

A first exemplary aluminum oxide coating method includes the sequential steps of: (a) loading the uncoated particles into a reactor, (b) applying a vaporous or gaseous aluminum precursor (e.g., TMA) to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(c) may be repeated one or more times to increase the total thickness of the aluminum oxide that enclose the solid core of the coated particles. The reactor pressure may be allowed to stabilize following step (a), step (b), and/or step (d). The reactor contents may be agitated prior to and/or during step (b), step (c), and/or step (c). A subset of vapor or gaseous content may be pumped out prior to step (c) and/or step (e).

A second exemplary aluminum oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous aluminum precursor (e.g., TMA), (c) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more aluminum oxide materials that enclose the solid core of the coated particles.

### Methods for Titanium Oxide Coating

In one aspect, the disclosure provides methods for preparing stable formulations comprising uncoated particles (e.g., drug particles and/or ingredient particles) encapsulated by titanium oxide coating.

The titanium oxide coating can be performed on a rotary powder coating chamber. The operating temperature can be 35-50° C. or about 50° C. For each titanium oxide coating process, one precursor may be introduced to reach a pressure of 0.3-2 torr for a hold time of 60 seconds, before nitrogen gas is used to purge the excess reactants and side products. The second precursor may then be introduced to reach a pressure of 2-8 torr for a hold time of 60 seconds, before nitrogen gas is used to purge the excess reactants and side products. This completes one coating cycle. Desired cycle numbers can be decided and titanium oxide coating can be coated in repeated cycles to obtain a desired thickness.

The titanium oxide coating can be applied using vapor phase deposition as described herein. The titanium precursor can be titanium tetrachloride (TiCl4), tetrakis(dimethylamino) titanium (TDMAT), tetrakis (diethylamino) titanium (TDEAT), or tetrakis(ethylmethylamino) titanium (TEMAT). The oxidant can be water or ozone. The titanium precursor may be titanium tetrachloride and the oxidant may be water.

A first exemplary titanium oxide coating method includes the sequential steps of: (a) loading the uncoated particles into a reactor, (b) applying a vaporous or gaseous titanium precursor (e.g., TiCl4) to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(c) may be repeated one or more times to increase the total thickness of the titanium oxide that enclose the solid core of the coated particles. The reactor pressure may be allowed to stabilize following step (a), step (b), and/or step (d). The reactor contents may be agitated prior to and/or during step (b), step (c), and/or step (c). A subset of vapor or gaseous content may be pumped out prior to step (c) and/or step (e).

A second exemplary titanium oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous titanium precursor (e.g., TiCl4), (c) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more titanium oxide materials that enclose the solid core of the coated particles.

### Methods for Zinc Oxide Coating

In one aspect, the disclosure provides methods for preparing stable formulations comprising uncoated particles (e.g., drug particles and/or ingredient particles) encapsulated by zinc oxide coating.

The first exemplary method includes the sequential steps of: (a) loading the uncoated particles into a reactor, (b) applying a vaporous or gaseous zinc precursor to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(e) can be repeated one or more times to increase the total thickness of the zinc oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

The second exemplary method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous zinc precursor, (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) can be repeated one or more times to increase the total thickness of the one or more zinc oxide materials that enclose the solid core of the coated particles.

The atomic layer coating process may comprise: (b1) loading the uncoated particles into a reactor; (b2) applying a vaporous or gaseous zinc precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas. Steps (b2)-(b5) can be performed two or more times to increase the total thickness of the zinc oxide layer before step (c) is performed.

The reactor pressure may be allowed to stabilize following step (b1), step (b2), and/or step (b4). The reactor contents may be agitated prior to and/or during step (b1), step (b3), and/or step (b5). A subset of vapor or gaseous content may be pumped out prior to step (b3) and/or step (b5). Step (b) may take place at a temperature between 45° C. and 55° C.

### Methods for Silicon Oxide Coating

In one aspect, the disclosure provides methods for preparing stable formulations comprising uncoated particles (e.g., drug particles and/or ingredient particles) encapsulated by silicon oxide coating.

The silicon oxide coating can be applied using vapor phase deposition as described herein. The silicon precursors can be silicon tetrachloride or 1,2-Bis(diisopropylamino)disilane (BDIPADS). The oxidant can be water or ozone.

A first exemplary silicon oxide coating method includes the sequential steps of: (a) loading the uncoated particles into a reactor, (b) applying a vaporous or gaseous silicon precursor (e.g., silicon tetrachloride, BDIPADS) to the substrate in the reactor, (c) performing one or more pump-purge cycles of the reactor using inert gas, (d) applying a vaporous or gaseous oxidant (e.g., ozone) to the substrate in the reactor, and (e) performing one or more pump-purge cycles of the reactor using inert gas. The sequential steps (b)-(e) may be repeated one or more times to increase the total thickness of the silicon oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

A second exemplary silicon oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous silicon precursor (e.g., silicon tetrachloride, BDIPADS), (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., ozone), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more silicon oxide materials that enclose the solid core of the coated particles.

Methods for Preparing a Coated Particle with Multiple Coating Layers

In some cases, the coating is applied using two or more different processes, for example, a silicon oxide coating process and an aluminum oxide coating process. The two or more different processes can be combined, e.g., to provide a distinct, inner aluminum oxide coating layer adjacent to the protein particle and a distinct, outer silicon oxide coating layer that encloses the particle, including the inner layer. In some cases, another coating layer can be applied surrounding the outer coating layer. Thus, there can be a distinct inner layer of a first metal or metalloid oxide, a distinct intermediate layer of a second metal or metalloid oxide and a third outer layer of the first (or a third) metalloid oxide. In general, the formation of such distinct coating layers entails at least 10 cycles of vapor phase deposition for each distinct coating layer, generating a coating layer that is about 2-100 nanometers thick.

### Methods for Preparing a Coated Particle by Supercycles

In some cases, the coating is applied using two or more different processes, for example, a zinc oxide coating process and an aluminum oxide coating process. The two or more different processes can be combined to provide a coating that include a ternary compound layer (e.g., an aluminum-zinc-oxide layer or AZO layer) that more closely resembles combinations of two different metal or metalloid oxides, as opposed to distinct multi-layer coatings having multiple, distinct metal or metalloid coating layers (described above). A so-called supercycle process is used to produce these ternary compound layers. In the supercycle process, only a very limited number of cycles (e.g., less than 10, 1-10, 2-10, 3-10, 1-5, 2-8, 2-8 cycles) are carried out with a first precursor before switching to a second precursor. Each supercycle includes a number of cycles with a first precursor and a number of cycles with a second precursor. The relatively frequent alteration in precursor during a supercycle process produces a coating that does not have distinct layers. Thus, a coating layer prepared by carrying out a small number (e.g., 1-10) of cycles using an aluminum oxide precursor alternating with a small number of cycles using a zinc oxide precursor can be referred to as an AZO layer. Importantly, the composition of the ternary compound layer can adjusted by having different numbers cycles with each precursor, e.g., alternating 3 cycles with an aluminum oxide precursor and 2 cycles with a zinc oxide precursor or alternating 2 cycles with an aluminum oxide precursor and 5 cycles with a zinc oxide precursor. when 2 or more supercycle steps are carried out, the number of cycles with each precursor do not need to remain the same. Thus, there can be a first supercycle having 3 cycles with an aluminum oxide precursor and 2 cycles with a zinc oxide precursor followed by a second supercycle with 2 cycles with an aluminum oxide precursor and 5 cycles with a zinc oxide precursor.

Of course, a supercycle coating process can be combined with a more conventional coating process. Thus, a particle can have an inner layer produced using a supercycle process and an outer layer that is a distinct layer (i.e., not a ternary compound layer). The two types of coating layers can be applied in reverse order to produce a particle having an inner layer that is a distinct layer (i.e., not a ternary compound layer) and outer layer that is a ternary compound layer produced using a supercycle process.

In one example, the disclosure provides methods to prepare a coated particle that has a core and at least one coating layer applied using supercycles, each supercycle having a first number of first cycles and a second number of second cycles. The methods include the sequential steps of: (a) providing uncoated particles; (b) performing a first number of first cycles; using first inorganic oxide precursor and (c) performing a second number of second cycles using a second inorganic oxide precursor, wherein the first and second inorganic oxide precursors are for forming different inorganic oxides (e.g., the first precursor can be aluminum oxide precursor and the second precursor can be zinc oxide precursor). The vaporous or gaseous oxidant used in the first and second cycles can be the same or different.

The step of performing a first number of first cycles (step (b)) comprises: (b1) applying a vaporous or gaseous first inorganic oxide precursor to the particles in the reactor by pulsing the vaporous or gaseous first inorganic oxide precursor into the reactor; (b2) performing one or more pump-purge cycles of the reactor using an inert gas; (b3) applying a vaporous or gaseous oxidant to the particles in the reactor by pulsing the oxidant into the reactor; and (b4) performing one or more pump-purge cycles of the reactor using an inert gas.

The step of performing a second number of second cycles (step (c)) comprises: (c1) applying a vaporous or gaseous second inorganic oxide precursor to the particles in the reactor by pulsing the vaporous or gaseous second inorganic oxide precursor into the reactor; (c2) performing one or more pump-purge cycles of the reactor using an inert gas; (c3) applying a vaporous or gaseous oxidant to the particles in the reactor by pulsing the oxidant into the reactor; and (c4) performing one or more pump-purge cycles of the reactor using an inert gas.

The steps (b)-(c) constitutes a supercycle. Steps (b)-(c) can be performed two or more times to increase the total thickness of the coating. The particles can be agitated prior to and/or during step (a). The reactor pressure can be allowed to stabilize following step (b1), step (b2), step (b3) and/or step (b4). The reactor pressure can be allowed to stabilize following step (c1), step (c2), step (c3) and/or step (c4).

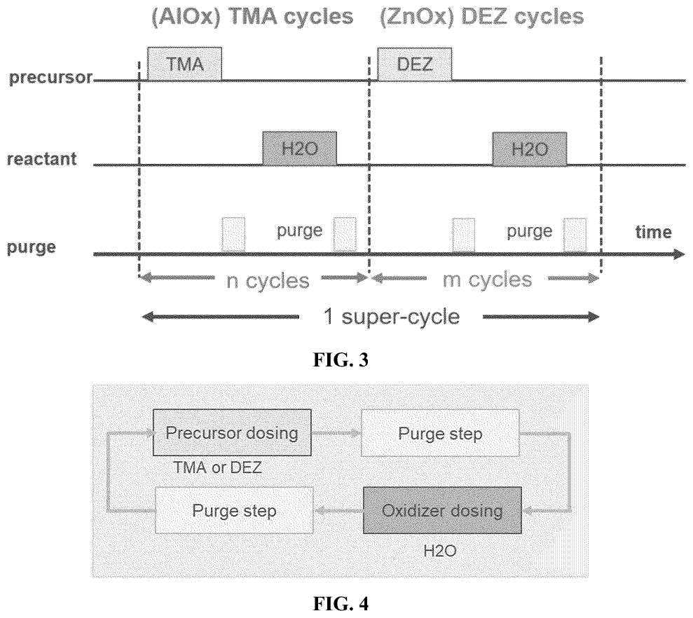

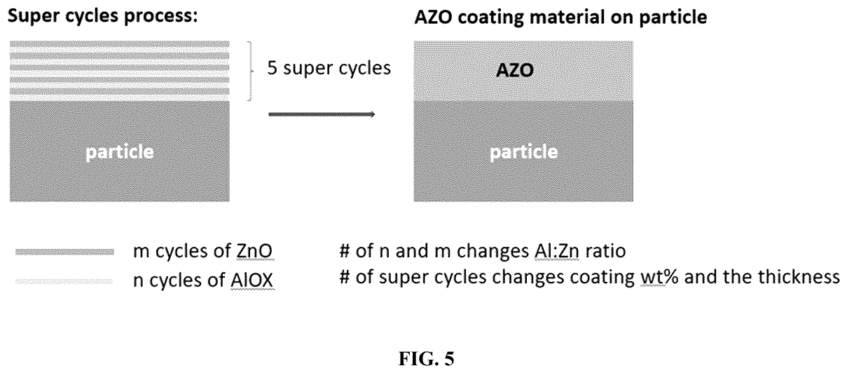

For example, aluminum oxide and zinc oxide precursors can be applied using supercycles to create an AZO layer (see, e.g., FIG. 3). The aluminum precursor can be trimethylaluminum (TMA). The zinc precursor can be diethylzinc (DEZ) or zinc tetrachloride.

As another example, aluminum oxide and silicon oxide precursors can be applied using supercycles to create an ASO layer. The aluminum precursor can be TMA. The silicon precursor can be SiCl4, Tris(tertpentoxy)silanol, diisopropylamino silane (DIPAS) or 1,2-Bis(diisopropylamino)disilane (BDIPADS).

### Coated Particles

In one aspect, the disclosure provides coated particles where a coating is applied to the uncoated particles (e.g., drug particles and/or ingredient particles) through the vapor phase deposition method described herein. The coated particles can be coated ingredient particles and/or coated drug particles. The coated particles may further contain one or more pharmaceutically acceptable excipients.

The coated ingredient particle may consist of an ingredient-containing core and an ingredient coating layer. The coated drug particle may consist of a drug-containing core and a drug coating layer. The thickness of the ingredient coating layer in the coated ingredient particle may be above 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the ingredient coating layer in the coated ingredient particle may be below 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the ingredient coating layer in the coated ingredient particle may be 50-300 nm, 80-200 nm, or 80-150 nm.

The thickness of the drug coating layer in the coated drug particle may be above 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the drug coating layer in the coated drug particle may be below 1 nm, 2 nm, 3 nm, 4 nm, 5 nm, 10 nm, 15 nm, 20 nm, 25 nm, 30 nm, 35 nm, 40 nm, 45 nm, 50 nm, 55 nm, 60 nm, 65 nm, 70 nm, 80 nm, 90 nm, 100 nm, 110 nm, 120 nm, 130 nm, 140 nm, 150 nm, or 200 nm. The thickness of the drug coating layer in the coated drug particle may be 5-100 nm, 10-50 nm, or 10-30 nm.

The coated particles can have reduced solid state reactions. The coated particles can have an improved flowability compared to uncoated particles. Applying the coating may improve the wettability and/or dispersibility of the uncoated particles. Applying the coating may improve the dispersibility, but not the wettability of the uncoated particles. Applying the coating may slow the release of the active ingredient in the uncoated particles.

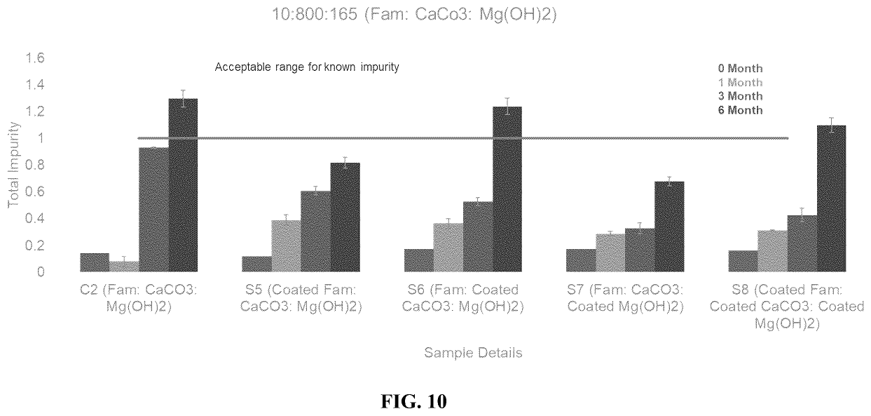

The amount of solid state reactions can be assessed by stability testing and/or impurity analysis. For example, the uncoated and coated particles can be first subject to storage for a given period of time (e.g., 6 months at room temperature) and then subject to impurities analysis (e.g., by HPLC). As another example, the uncoated and coated particles can be subject to (1) accelerated stability testing (e.g., storage at 40° C. and 75% relative humidity) and (2) United States Pharmacopcia (USP) method for high pressure liquid chromatography (HPLC). The USP provides a global and extensive source for upholding medicine quality, publishing over 5000 standards for medicines that cover both active and inactive pharmaceutical ingredients. Their monographs describe tests to validate medicines, meeting the criteria and quality expectations required across the pharmaceutical space. One such document, the USP General Chapter <621> “Chromatography”, upholds the rigorous standards required in chromatographic techniques. The uncoated and coated particles can be subject to accelerated stability testing (e.g., storage at 40° C. and 75% relative humidity) for a suitable period of time, e.g., 1 day, 2 days, 3 days, 5 days, 1 week, 2 weeks, 1 month, 2 months, 3 months, 4 months, 5 months, 6 months, 1 year or 2 years. FIG. 10 shows exemplary results of impurity analysis for coated and uncoated drug and ingredient particles after accelerated stability testing. Comparing to the uncoated particles, the coated particles can have reduced amounts of impurities. The coating methods described herein may reduce the amount of impurities by more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%.

The structure of the ingredient and/or the drug can be assessed by X-Ray Diffraction (XRD) analysis. The structure of ingredient and/or the drug can be assessed by Fourier-transform infrared (FTIR) analysis. The composition of the coated particles can be assessed by Thermogravimetric Analysis (TGA) analysis. The coating process may cause no significant structural change in the ingredient and/or the drug.

The dissolution or ingredient/drug release of the coated particles can be assessed by an in vitro release over time (dissolution) analysis. For example, the dissolution or ingredient/drug release of the coated particles can be assessed by HPLC analysis. The dissolution can be assessed by dissolving the coated particles in a sodium phosphate buffer solution (PBS) (e.g., pH 7.2, with or without surfactant) at 37° C., with a stirring of 100 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours. The coated particles can have a reduced dissolution rate compared to uncoated particles. For example, the dissolution rate of the coated particles may be at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, lower than the dissolution of uncoated particles. The uncoated particles may exhibit an immediate release profile.

The morphology of the coated particles can be assessed by Transmission Electron Microscopy (TEM) analysis and/or Scanning Electron Microscopy (SEM) analysis. The coating process may lead to no obvious morphology change.

### Stable Formulations

In one aspect, the disclosure is related to stable formulations that comprise (1) coated ingredient particles and (2) optionally coated drug particles. The stable formulation can comprise a dry blend of (1) coated ingredient particles and (2) optionally coated drug particles. The coating in the coated ingredient particles may be the same as the coating in the coated drug particles.

The coating (e.g., aluminum oxide coating) described herein can act as a physical barrier and reduce interaction between the drug (e.g., famotidine) particles and the coated ingredient (e.g., CaCO₃ or Mg(OH)₂) particles in the formulation. The coating can also act as a physical barrier to reduce between a first ingredient (e.g., CaCO₃) particle and a second ingredient (e.g., Mg(OH)₂) particle. The coating can also act a physical barrier to reduce exposure to water/moisture by the coated drug particles and/or the coated ingredient particles. The coating can also act as a physical barrier to reduce exposure to oxygen/oxidants by the coated drug particles and/or the coated ingredient particles. The coating can reduce hydrolyzation and/or oxidation of the coated drug particles and/or the coated ingredient particles. The coating described herein may act as a physical barrier to reduce exposure of a hygroscopic ingredient (e.g., Mg(OH)₂) in the formulation to the environment.

Both the ingredient coating layer and the drug coating layer can be prepared by supercycles as described herein. Both the ingredient coating layer and the drug coating layer can comprise an AZO coating. The Al/Zn ratio (wt/wt) of the ingredient coating layer in the coated ingredient particle may be more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25, more than 0.3, more than 0.35, more than 0.4, more than 0.45, more than 0.5, more than 0.6, more than 0.7, more than 0.9, or more than 0.9. The Al/Zn ratio of the ingredient coating layer in the coated ingredient particle may be less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. The Al/Zn ratio of the ingredient coating layer in the coated ingredient particle may be 0.1-0.5, 0.1-0.4, 0.1-0.35, 0.15-0.35 or 0.25-0.35.

The Al/Zn ratio (wt/wt) of the drug coating layer in the coated drug particle may be more than 0.01, more than 0.02, more than 0.03, more than 0.04, more than 0.05, more than 0.06, more than 0.07, more than 0.08, more than 0.09, more than 0.1, more than 0.11, more than 0.12, more than 0.13, more than 0.14, more than 0.15, more than 0.2, more than 0.25, more than 0.3, more than 0.35, more than 0.4, more than 0.45, more than 0.5, more than 0.6, more than 0.7, more than 0.9, or more than 0.9. The Al/Zn ratio of the drug coating layer in the coated drug particle may be less than 0.01, less than 0.02, less than 0.03, less than 0.04, less than 0.05, less than 0.06, less than 0.07, less than 0.08, less than 0.09, less than 0.1, less than 0.11, less than 0.12, less than 0.13, less than 0.14, less than 0.15, less than 0.2, less than 0.25, less than 0.3, less than 0.35, less than 0.4, less than 0.45, less than 0.5, less than 0.6, less than 0.7, less than 0.9, or less than 0.9. The Al/Zn ratio of the drug coating layer in the coated drug particle may be 0.3-0.9, 0.3-0.7, 0.4-0.7, or 0.4-0.6.

The ratio between (1) coated ingredient particles and (2) drug particles can be more than 0.0.1, 0.02, 0.05, 0.1, 0.2. 0.3. 0.4, 0.5, 0.6, 0.7, 0.8, 0.9, 1, 1.5, 2, 3, 4, 5, 6, 7, 8, 9, 10, 20, 50, or 100. The ratio between (1) coated ingredient particles and (2) drug particles can be less than 0.0.1, 0.02, 0.05, 0.1, 0.2. 0.3. 0.4, 0.5, 0.6, 0.7, 0.8, 0.9, 1, 1.5, 2, 3, 4, 5, 6, 7, 8, 9, 10, 20, 50, or 100. The coated ingredient particles may constitute more than 1%, 2%, 3%, 4%, 5%, 6%, 7%, 8%, 9%, 10%, 20%, 30%, 40%, 50%, 60%, 70%, 80%, 90% of the stable formulation by weight. The coated ingredient particles may constitute less than 1%, 2%, 3%, 4%, 5%, 6%, 7%, 8%, 9%, 10%, 20%, 30%, 40%, 50%, 60%, 70%, 80%, 90% of the stable formulation by weight. The drug particles may constitute more than 1%, 2%, 3%, 4%, 5%, 6%, 7%, 8%, 9%, 10%, 20%, 30%, 40%, 50%, 60%, 70%, 80%, 90% of the stable formulation by weight. The drug particles may constitute less than 1%, 2%, 3%, 4%, 5%, 6%, 7%, 8%, 9%, 10%, 20%, 30%, 40%, 50%, 60%, 70%, 80%, 90% of the stable formulation by weight.

The pharmaceutical compositions can be formulated in any suitable manner known in the art. For example, the pharmaceutical compositions can be in the form of tablets, capsules, powders, microparticles, granules, syrups, suspensions, solutions, nasal spray, transdermal patches, injectable solutions, or suppositories.

Pharmaceutical compositions are formulated to be compatible with their intended route of administration (e.g., oral, intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents (e.g., benzyl alcohol or methyl parabens, chlorobutanol, phenol, ascorbic acid, and thimerosal), antioxidants (e.g., ascorbic acid and sodium bisulfite), chelating agents (e.g., ethylenediaminetetraacetic acid), buffers (e.g., acetates, citrates, and phosphates), and isotonic agents (e.g., sugars (e.g., dextrose), polyalcohols (e.g., mannitol or sorbitol), and salts (e.g., sodium chloride)), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating (e.g., lecithin) or a surfactant. Controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Pharmaceutically acceptable carriers, adjuvants and vehicles that can be used in the pharmaceutical compositions of the present disclosure include ion exchangers, alumina, aluminum stearate, lecithin, serum proteins (e.g., human serum albumin), buffer substances (e.g., phosphates, glycine), sorbic acid, potassium sorbate, partial glyceride mixtures of saturated vegetable fatty acids, water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or formulations can contain the coated particles described herein in the range of 0.001% to 100% (e.g., 0.1-95%, 20-80%, or 75-85%) with the balance made up from the suitable pharmaceutically acceptable excipients.

The coating may simply the formulation process or other manufacturing process of a pharmaceutical composition. For example, the coating may eliminate the need to include additional detergents in the final formulation.

### EXAMPLES

### Example 1: Preparation of Stable Formulation

To reduce solid state reactions and drug degradation, the drug (famotidine) particles and/or ingredient (calcium carbonate and/or magnesium hydroxide) particles were coated with an inorganic oxide coating. Specifically, uncoated particles (famotidine, calcium carbonate and/or magnesium hydroxide) were coated with aluminum oxide at 50° C. for 40 cycles following the methods described herein. The vaporous or gaseous metal precursor is tri-methyl aluminum (TMA), the byproduct gaseous methane is formed after TMA reacts with exposed hydroxyl groups on the particles or on surface of the coated particles, and the oxidant is water vapor.

The method comprised the sequential steps of:
(a) loading uncoated particles (famotidine, calcium carbonate and/or magnesium hydroxide) into a reactor;
(b) reducing the reactor pressure to less than 1 Torr;
(c) agitating the reactor contents until the reactor contents has a desired water content by performing residual gas analysis (RGA) to monitor levels of water vapor in the reactor;
(d) pressurizing the reactor to at least 1 Torr by adding a vaporous or gaseous TMA;
(e) allowing the reactor pressure to stabilize;
(f) agitating the reactor contents;
(g) pumping out a subset of vapor or gaseous content, including gaseous methane and unreacted TMA, and determining when to stop pumping by performing RGA to monitor levels of gaseous methane and unreacted TMA in the reactor.
(h) performing a sequence of pump-purge cycles on the reactor using nitrogen gas;
(i) pressuring the reactor to at least 1 Torr by adding water vapor;
(j) allowing the reactor pressure to stabilize;
(k) agitating the reactor contents;
(l) pumping out a subset of vapor or gaseous content, including water vapor, and determining when to stop pumping by performing RGA to monitor levels of water vapor in the reactor;
(m) performing a sequence of pump-purge cycles on the reactor using nitrogen gas.

Additionally, the steps of (b)-(m) were repeated more than once to increase the total thickness of the aluminum oxide that enclose said solid core.

### Example 2: Characterization of Uncoated and Coated Drug Particles

Pre-coating characterization was performed on uncoated famotidine drug particles before coating. First, the uncoated famotidine drug particles were subject to laser diffraction particle size analysis (PSA) to determine the D50 (the corresponding particle size value when the cumulative distribution percentage reaches 50%).

Second, the uncoated drug particles were subject to BET specific surface area analysis, to determine the specific surface area. Specifically, samples were first prepared by drying, with a flow of inert gas or vacuum atmosphere, to clear the surface of any contaminants. The samples were introduced to cryogenic temperature to allow a probe gas to physically adsorb to the surface of the samples. The volume of probe gas adsorbed was measured to determine the quantity of gas required to cover the surface of the sample. The Brunauer, Emmett and Teller (BET) theory is then applied to the adsorption data to generate a specific surface area, reported in units of area per mass of sample (m²/g).

Third, the uncoated drug particles were subject to flowability analysis. A powder rheometer can be used to measure dynamic, bulk, and shear properties in addition to wall friction measurements. The variety of testing techniques allows for the measurement of a powder in several different powder “states”: dynamic, static, or under a stressed condition. The powder rheometer measures the energy needed to create specific flow conditions by passing a blade through a conditioned powder sample under various testing methodologies. Since Powder Flowability can be dramatically influenced by entraining air during transfer or by compacting the sample into a container, the testing methodology incorporates techniques to evaluate as well as limit these conditions.

Finally, the appearance and morphology of the uncoated particles were observed. The morphology of the uncoated particles were assessed by scanning electron microscopy (SEM).

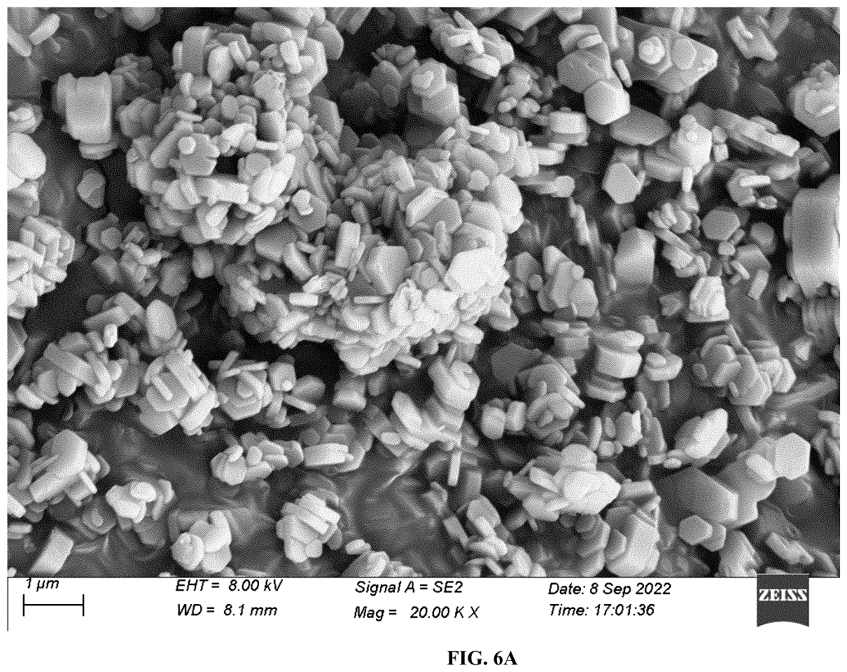

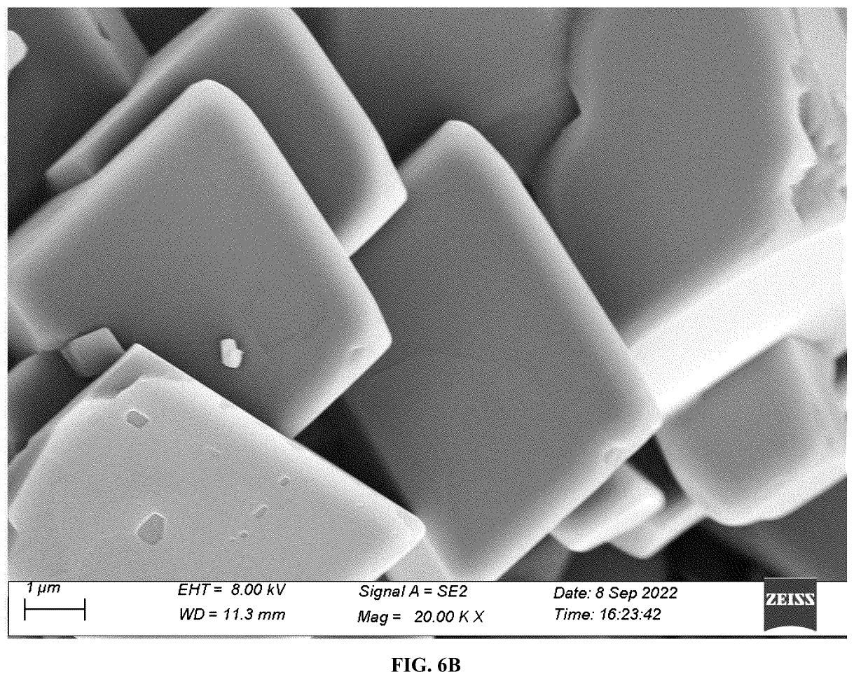

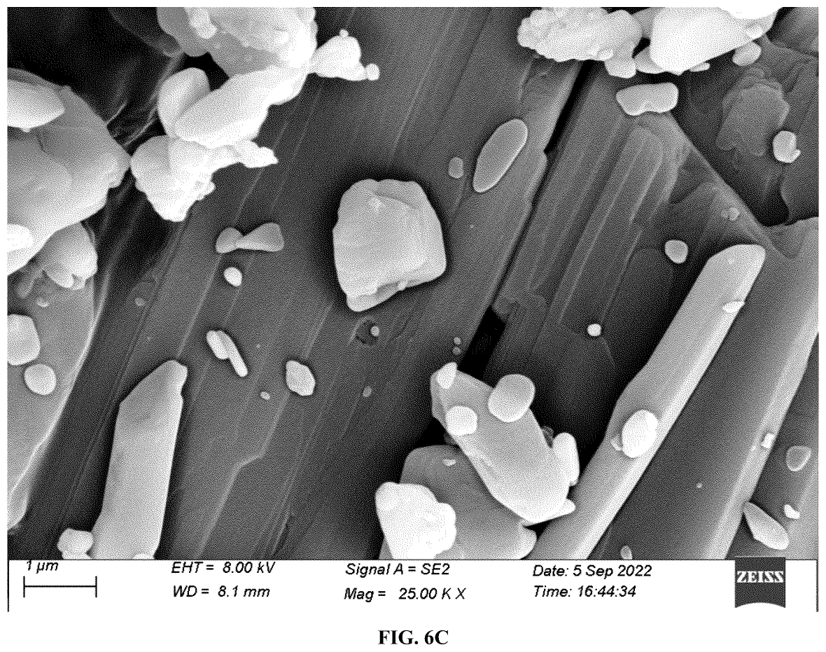

The pre-coating characterization results are shown in the below table. The morphology of uncoated famotidine drug particles are shown in FIGS. 6A-6C.

TABLE 1
Pre-coating characterization data

| | PSA | | BET | Flow | |
|---|---|---|---|---|---|
| Sample Name | (D50) | | (m2/g) | Factor | Appearance |
| Magnesium hydroxide | ~1.5 | um | 6.2042 | 5 | white |
| Calcium carbonate | 11 | um | 0.1851 | 3.2 | White |
| Famotidine | ~6 | um | 1.3136 | — | white |

Post-coating characterization was performed on coated famotidine drug particles after coating. First, the coated famotidine drug particles were subject to laser diffraction particle size analysis (PSA) to determine the D50.

Second, the coated drug particles were subject to Inductively Coupled Plasma Atomic Emission Spectroscopy (ICP-AES) to determine the amount of aluminum (Al).

Finally, the appearance and morphology of the coated particles were observed. The morphology of the uncoated particles were assessed by scanning electron microscopy (SEM).

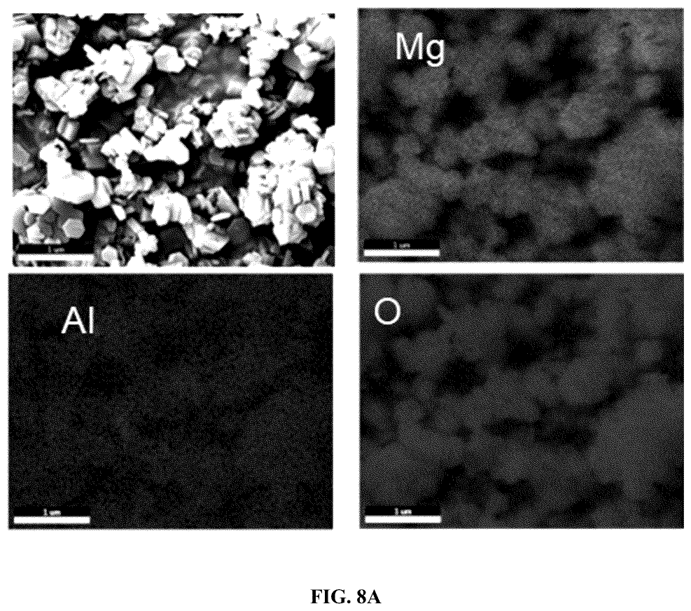

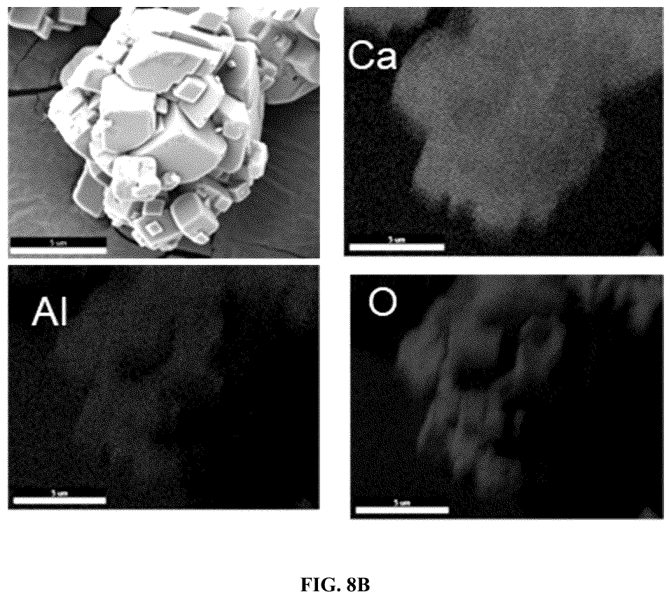

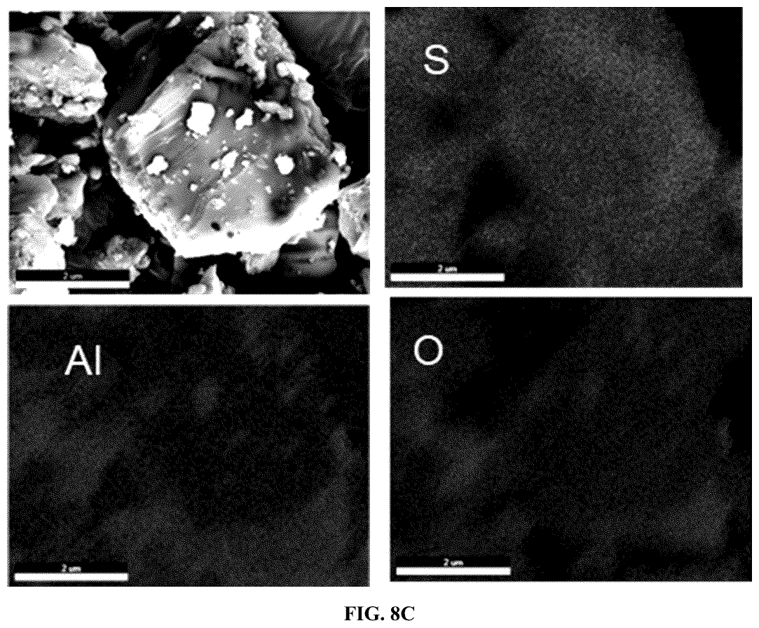

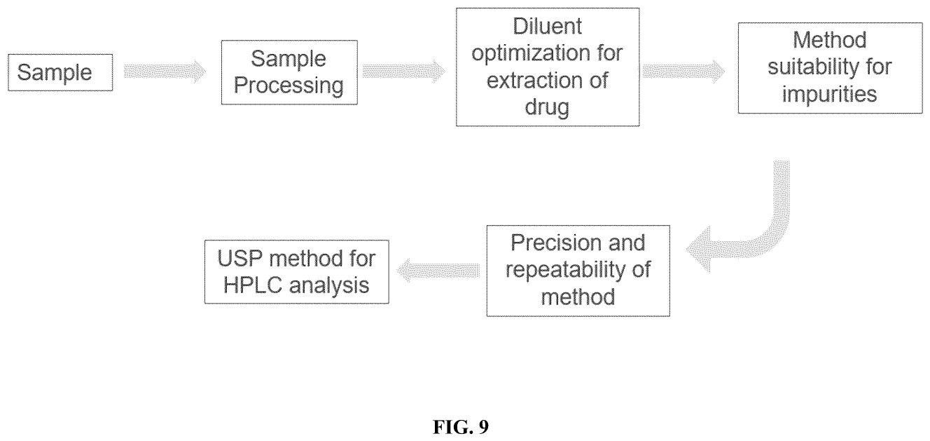

The results of post-coating characterization are shown in the below table. The morphology of coated famotidine drug particles are shown in FIGS. 8A-8C. FIG. 8A shows magnesium hydroxide particles, showing a uniform aluminum oxide coating was applied to the magnesium hydroxide particles. FIG. 8B shows calcium carbonate particles, showing a uniform aluminum oxide coating was applied to the calcium carbonate particles. FIG. 8C shows famotidine particles, showing a uniform aluminum oxide coating was applied to the famotidine particles.

TABLE 2
Post-coating characterization data

| | | | ICP-AES | |
|---|---|---|---|---|
| Sample Name | PSA (D50) | | (for Al) | Appearance |
| Coated Mg(OH)2 (WO105) | 2.8 | um | 1.103% | White |
| Coated CaCO3 (WO107) | 11.5 | um | 0.253% | White |
| Coated Fam (WO108) | 7 | um | 1.641% | White |

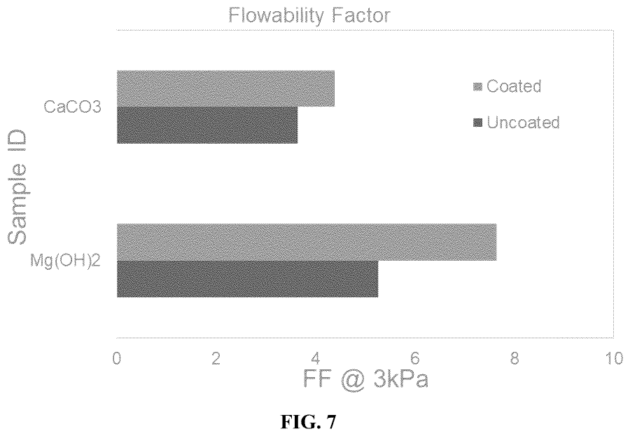

Further, uncoated and coated ingredient (calcium carbonate and magnesium hydroxide) particles were subject to flowability analysis. Flowability analysis results are shown in FIG. 7. As shown in FIG. 7, comparing to uncoated ingredient particles, the coated ingredient particles showed increased flowability.

### Example 3: Preparation of Stable Formulation

Five different pharmaceutical formulations (Control-2, S-5, S-6, S-7, and S-8) containing uncoated and/or coated particles were prepared according to the recipes in the table below. These formulations were pressed into tablets.

TABLE 3
different pharmaceutical formulations

| Sample code | Combination | Ratio (%) |
|---|---|---|
| C-2 | API + CaCO3 + Mg(OH)2 | 10:800:165 |
| S-5 | Coated API + CaCO3 + Mg(OH)2 | 10:800:165 |
| S-6 | API + Coated CaCO3 + Mg(OH)2 | 10:800:165 |
| S-7 | API + CaCO3 + Coated Mg(OH)2 | 10:800:165 |
| S-8 | Coated API + Coated CaCO3 + Coated | 10:800:165 |
| | Mg(OH)2 | |

### Example 4: Stability Testing and Impurity Analysis

The pharmaceutical formulations prepared in Example 3 were subject to storage at 40° C. and 75% relative humidity for 6 months. Samples were taken at 0 month, 1 month, 2 months, 3 months, and 6 months after storage, and were subject to HPLC analysis following the parameters in the below table. The results of the stability testing and impurity analysis are shown in FIG. 10. As shown in FIG. 10, formulation C2 (uncoated particles) had the highest impurity levels after storage. By comparison, formulations with coated particles (formulation S5, S6, S7, and S8) had reduced impurity levels after storage.

TABLE 4
HPLC conditions

| | |
|---|---|
| Column | C18 column |
| Mobile phase | 13:87 (v/v) Acetonitrile-0.1M dihydrogen |
| | phosphate buffer containing 0.2% |
| | triethylamine (pH 3.0); |
| Flow rate | 1 mL/min |
| Detection wavelength | 265 nm |

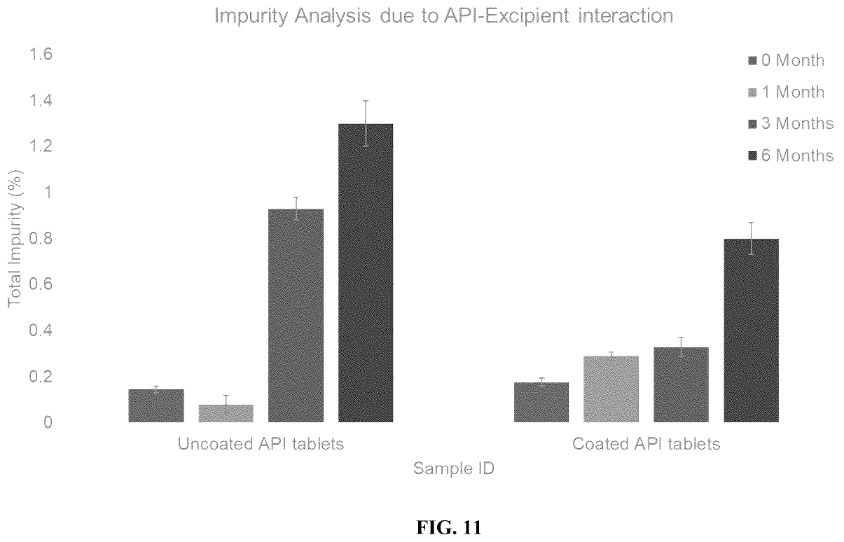

Further, the pharmaceutical formulations prepared in Example 3 were formulated into tablets and were subject to storage at 40° C. and 75% relative humidity for 6 months. Samples were taken at 0 month, 1 month, 2 months, 3 months, and 6 months after storage, and were subject to HPLC analysis. The results of the stability testing and impurity analysis are shown in FIG. 11. As shown in FIG. 11, uncoated particles (formulation C2) had the high impurity levels after storage. By comparison, formulations with coated particles (formulation S5) had reduced impurity levels after storage.

### Example 5: Dissolution of Uncoated and Coated Famotidine

To assess how the coating affects the dissolution of the coated drug particles, uncoated and coated drug particles were subject to dissolution analysis. Briefly, the uncoated and coated drug (famotidine) particles were dissolved in a sodium phosphate buffer solution (PBS) (pH 7.2) at 37° C., with a stirring of 100 revolutions per minute (RPM) for a total of 1400 minutes. Samples were taken at various time points and were analyzed by HPLC to assess the amount of dissolved famotidine in the solution.

The results are shown in FIG. 13. As shown in FIG. 13, comparing to uncoated drug particles, the coated drug particles showed a slightly slower drug release. But the total amount of drug release is largely the same.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A pharmaceutical composition, comprising:
(a) a coated ingredient particle consisting of an ingredient-containing core comprising an ingredient, and an ingredient coating layer enclosing the ingredient-containing core; and
(b) a drug particle,
wherein the ingredient coating layer comprises an inorganic oxide that comprises aluminum, zinc, silicon, and/or titanium.

2. The pharmaceutical composition of claim 1, wherein the drug particle is a coated drug particle that comprises a drug-containing core comprising a drug and a drug coating layer enclosing the drug-containing core, wherein the drug coating layer comprises an inorganic oxide that comprises aluminum, zinc, silicon, and/or titanium.

3. The pharmaceutical composition of claim 2, wherein the ingredient coating layer in the coated ingredient particle is the same as the drug coating layer in the coated drug particle.

4. The pharmaceutical composition of claim 1, wherein the ingredient coating layer is 0.1 nm-120 nm thick.

5.-8. (canceled)

9. The pharmaceutical composition of claim 1, wherein the ingredient-containing core has a D50 on a volume average basis of 100 nm-30 micrometers.

10. The pharmaceutical composition of claim 1, wherein the ingredient-containing core has a median particle size, on a volume average basis, between 0.1 μm and 20 μm.

11. The pharmaceutical composition of claim 1, wherein the drug is an organic molecule.

12. (canceled)

13. The pharmaceutical composition of claim 1, wherein the ingredient is a pharmaceutically acceptable excipient or a second drug that is different from the drug in the drug particle.

14. The pharmaceutical composition of claim 1, wherein the ingredient and the drug can undergo a solid state reaction.

15. The pharmaceutical composition of claim 1, wherein the ingredient is hygroscopic.

16. The pharmaceutical composition of claim 1, wherein the ingredient is calcium carbonate or magnesium hydroxide.

17. (canceled)

18. The pharmaceutical composition of claim 1, comprising a dry mix of the coated ingredient particle and the drug particle.

19. (canceled)

20. The pharmaceutical composition of claim 1, wherein the ingredient coating layer stabilizes the formulation, leading to less impurities after 1 month of storage at 40° C. and 75% relative humidity.

21. (canceled)

22. (canceled)

23. A method of preparing a pharmaceutical composition, the method comprising the sequential steps of:
(a) loading particles comprising an ingredient into a chamber of a reactor, wherein the ingredient is a pharmaceutically acceptable excipient;
(b1) applying a vaporous or gaseous precursor to the particles in the reactor by pulsing the vaporous or gaseous aluminum precursor into the reactor;
(b2) purging using an inert gas;
(b3) applying a vaporous or gaseous oxidant to the particles in the reactor by pulsing the oxidant into the reactor;
(b4) purging using an inert gas;
(c) repeating steps (b1)-(b4) at least once to create a coated ingredient particle; and
(d) mixing the coated ingredient particle with a drug particle.

24. (canceled)

25. The method of claim 23, wherein the drug particle is a coated drug particle that is prepared by a method comprising the sequential steps of:
(a) loading particles comprising a drug into a chamber of a reactor;
(b1) applying a vaporous or gaseous precursor to the particles in the reactor by pulsing the vaporous or gaseous aluminum precursor into the reactor;
(b2) purging using an inert gas;
(b3) applying a vaporous or gaseous oxidant to the particles in the reactor by pulsing the oxidant into the reactor;
(b4) purging using an inert gas; and
(c) repeating steps (b1)-(b4) at least once to create the coated drug particle.

26. (canceled)

27. The method of claim 23, wherein the resulting pharmaceutical composition comprises (a) a coated ingredient particle consisting of an ingredient-containing core comprising an ingredient, and an ingredient coating layer enclosing the ingredient-containing core; and (b) a coated drug particle consisting of a drug-containing core comprising a drug, and a drug coating layer enclosing the drug-containing core.

28.-37. (canceled)

38. A method of preparing a pharmaceutical composition, the method comprising the sequential steps of:
(a) loading particles ingredient containing core comprising an ingredient into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) purging using an inert gas;
(b3) applying a vaporous or gaseous oxidant to the particles in the reactor by pulsing the oxidant into the reactor;
(b4) purging using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) purging using an inert gas;
(c3) applying a vaporous or gaseous oxidant to the particles in the reactor by pulsing the oxidant into the reactor;
(c4) purging using an inert gas; and
(d) repeating steps (b)-(c) at least once to create a coated ingredient particle; and
(e) mixing the coated ingredient particle with a drug particle,
wherein, the first inorganic oxide precursor and second inorganic oxide precursor are different and the first number is an integer selected from 1-10, and the second number is an integer selected from 1-10.

39. (canceled)

40. The method of claim 38, wherein the drug particle is a coated drug particle prepared by a method comprising the sequential steps of:
(a) loading particles comprising a drug into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) purging using an inert gas;
(b3) applying a vaporous or gaseous oxidant to the particles in the reactor by pulsing the oxidant into the reactor;
(b4) purging using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) purging using an inert gas;
(c3) applying a vaporous or gaseous oxidant to the particles in the reactor by pulsing the oxidant into the reactor;
(c4) purging using an inert gas; and
(d) repeating steps (b)-(c) at least once to create a coated drug particle,
wherein, the first inorganic oxide precursor and second inorganic oxide precursor are different and the first number is an integer selected from 1-10, and the second number is an integer selected from 1-10.

41. (canceled)

42. The method of claim 38, wherein the resulting pharmaceutical composition comprises (a) a coated ingredient particle consisting of an ingredient-containing core comprising an ingredient, and an ingredient coating layer enclosing the ingredient-containing core; and (b) a coated drug particle consisting of a drug-containing core comprising a drug, and a drug coating layer enclosing the drug-containing core.

43.-72. (canceled)

73. The pharmaceutical composition of claim 1, wherein the ingredient is a pharmaceutically acceptable excipient.
